SEQUENCE LISTING (65 sequences)

SEQ ID NO: 1 - protein, 249 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQYRKLAWTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGSIFNGYDVLDNWGQGTLVTVSS

SEQ ID NO: 2 - DNA, 747 bp, Homo sapiens
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagtacaggaagctcgcatggactttcggccagggcaccaaactggagatcaagcgtggtggaggaggtagcggaggaggcgggagcggtggaggtggctctggaggtggcggaagccaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggggttctattttcaacggttacgacgttctggacaactggggccagggcacactagtgaccgtgtccagc

SEQ ID NO: 3 - protein, 121 aa, Homo sapiens
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGSIFNGYDVLDNWGQGTLVTVSS

SEQ ID NO: 4 - protein, 108 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQYRKLAWTFGQGTKLEIKR

SEQ ID NO: 5 - protein, 244 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQALVVTPFTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARFGMLDNWGQGTLVTVSS

SEQ ID NO: 6 - DNA, 735 bp, Homo sapiens
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcaggctcttgtggtgacgccgttcactttcggccagggcaccaaactggagatcaagcgtggtggaggaggtagcggaggaggcgggagcggtggaggtggctctggaggtggcggaagccaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggttttagggtatgctggacaactggggccagggcacactagtgaccgtgtccagc

SEQ ID NO: 7 - protein, 115 aa, Homo sapiens
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARFGMLDNWGQGTLVTVSS

SEQ ID NO: 8 - protein, 109 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQALVVTPFTFGQGTKLEIKR

SEQ ID NO: 9 - protein, 245 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQRLTPSPFTFGQGTKLEIKRGGGGSGGGGSGGGGSGGGGSQVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARNTALLDNWGQGTLVTVSS

SEQ ID NO: 10 - DNA, 735 bp, Homo sapiens
gacatccagatgacccagagccctagctcactgagcgccagcgtgggcgacagggtgaccattacctgctccgccagccaggacatcagcaactacctgaactggtaccagcagaagcccggcaaggcccccaagctgctgatctactacacctccaacctgcactccggcgtgcccagcaggttcagcggaagcggcagcggcaccgatttcaccctgaccatctccagcctgcagcccgaggacttcgccacctactactgccagcagcgtttgacgccgtctccgttcactttcggccagggcaccaaactggagatcaagcgtggtggaggaggtagcggaggaggcgggagcggtggaggtggctctggaggtggcggaagccaggtgcagctggtccagagcggcgccgaagtgaagaagcccggcagctccgtgaaagtgagctgcaaggccagcggcggcaccttcagcaactactggatgcactgggtgaggcaggcccccggacagggcctggagtggatgggcgccacctacaggggccacagcgacacctactacaaccagaagttcaagggccgggtgaccatcaccgccgacaagagcaccagcaccgcctacatggaactgagcagcctcaggagcgaggacaccgctgtgtattactgcgccaggaatacggctttgctggacaactggggccagggcacactagtgaccgtgtccagc

SEQ ID NO: 11 - protein, 116 aa, Homo sapiens
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARNTALLDNWGQGTLVTVSS

SEQ ID NO: 12 - protein, 108 aa, Homo sapiens
DIQMTQSPSSLSASVGDRVTITCSASQDISNYLNWYQQKPGKAPKLLIYYTSNLHSGVPSRFSGSGSGTDFTLTISSLQPEDFATYYCQQRLTPSPFTFGQGTKLEIK

SEQ ID NO: 13 - protein, 26 aa, Homo sapiens [sequence omitted]

SEQ ID NO: 14 - protein, 6 aa, Homo sapiens
QDISNY

SEQ ID NO: 15 - protein, 17 aa, Homo sapiens
LNWYQQKPGKAPKLLIY

SEQ ID NO: 16 - protein, 4 aa, Homo sapiens
  MISC_FEATURE (4)..(4): Xaa is not existed, just for finishing the sequence listing
YTSX

SEQ ID NO: 17 - protein, 36 aa, Homo sapiens
NLHSGVPSRFSGSGSGTDFTLTISSLQPEDFATYYC

SEQ ID NO: 18 - protein, 9 aa, Homo sapiens
QQYRKLAWT

SEQ ID NO: 19 - protein, 11 aa, Homo sapiens
FGQGTKLEIKR

SEQ ID NO: 20 - protein, 25 aa, Homo sapiens [sequence omitted]

SEQ ID NO: 21 - protein, 8 aa, Homo sapiens
GGTFSNYW

SEQ ID NO: 22 - protein, 17 aa, Homo sapiens
MHWVRQAPGQGLEWMGA

SEQ ID NO: 23 - protein, 8 aa, Homo sapiens
TYRGHSDT

SEQ ID NO: 24 - protein, 38 aa, Homo sapiens
YYNQKFKGRVTITADKSTSTAYMELSSLRSEDTAVYYC

SEQ ID NO: 25 - protein, 14 aa, Homo sapiens
ARGAIYNGYDVLDN

SEQ ID NO: 26 - protein, 11 aa, Homo sapiens
WGQGTLVTVSS

SEQ ID NO: 27 - protein, 26 aa, Homo sapiens [sequence omitted]

SEQ ID NO: 28 - protein, 6 aa, Homo sapiens
QDISNY

SEQ ID NO: 29 - protein, 17 aa, Homo sapiens
LNWYQQKPGKAPKLLIY

SEQ ID NO: 30 - protein, 4 aa, Homo sapiens
  MISC_FEATURE (4)..(4): Xaa is not existed, just for finishing the sequence listing
YTSX

SEQ ID NO: 31 - protein, 36 aa, Homo sapiens; identical to SEQ ID NO: 17

SEQ ID NO: 32 - protein, 10 aa, Homo sapiens
QQALVVTPFT

SEQ ID NO: 33 - protein, 11 aa, Homo sapiens
FGQGTKLEIKR

SEQ ID NO: 34 - protein, 25 aa, Homo sapiens [sequence omitted]

SEQ ID NO: 35 - protein, 8 aa, Homo sapiens
GGTFSNYW

SEQ ID NO: 36 - protein, 17 aa, Homo sapiens
MHWVRQAPGQGLEWMGA

SEQ ID NO: 37 - protein, 8 aa, Homo sapiens
TYRGHSDT

SEQ ID NO: 38 - protein, 38 aa, Homo sapiens; identical to SEQ ID NO: 24

SEQ ID NO: 39 - protein, 8 aa, Homo sapiens
ARFGMLDN

SEQ ID NO: 40 - protein, 11 aa, Homo sapiens
WGQGTLVTVSS

SEQ ID NO: 41 - protein, 26 aa, Homo sapiens [sequence omitted]

SEQ ID NO: 42 - protein, 6 aa, Homo sapiens
QDISNY

SEQ ID NO: 43 - protein, 17 aa, Homo sapiens
LNWYQQKPGKAPKLLIY

SEQ ID NO: 44 - protein, 4 aa, Homo sapiens
  MISC_FEATURE (4)..(4): Xaa is not existed, just for finishing the sequence listing
YTSX

SEQ ID NO: 45 - protein, 36 aa, Homo sapiens; identical to SEQ ID NO: 17

SEQ ID NO: 46 - protein, 10 aa, Homo sapiens
QQRLTPSPFT

SEQ ID NO: 47 - protein, 11 aa, Homo sapiens
FGQGTKLEIKR

SEQ ID NO: 48 - protein, 25 aa, Homo sapiens [sequence omitted]

SEQ ID NO: 49 - protein, 8 aa, Homo sapiens
GGTFSNYW

SEQ ID NO: 50 - protein, 17 aa, Homo sapiens
MHWVRQAPGQGLEWMGA

SEQ ID NO: 51 - protein, 8 aa, Homo sapiens
TYRGHSDT

SEQ ID NO: 52 - protein, 38 aa, Homo sapiens; identical to SEQ ID NO: 24

SEQ ID NO: 53 - protein, 9 aa, Homo sapiens
ARNTALLDN

SEQ ID NO: 54 - protein, 11 aa, Homo sapiens
WGQGTLVTVSS

SEQ ID NO: 55 - DNA, 60 bp, Artificial Sequence
  Synthesized
gtgtattactgcgccarggstkctrtttwcracgsttwcgamrttctggacaactggggc

SEQ ID NO: 56 - DNA, 48 bp, Artificial Sequence
  Synthesized
tactactgccagcagtwcargargmtcscatkgactttcggccagggc

SEQ ID NO: 57 - DNA, 52 bp, Artificial Sequence
  Synthesized
  misc_feature (24)..(25): n is a, c, g, or t
  misc_feature (27)..(28): n is a, c, g, or t
ccgctgtgtattactgcgccargnnknnkgstwtkctggacaactggggcca

SEQ ID NO: 58 - DNA, 58 bp, Artificial Sequence
  Synthesized
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
ccgctgtgtattactgcgccargnnknnknnknnkgstwtkctggacaactggggcca

SEQ ID NO: 59 - DNA, 73 bp, Artificial Sequence
  Synthesized
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
  misc_feature (36)..(37): n is a, c, g, or t
  misc_feature (39)..(40): n is a, c, g, or t
  misc_feature (42)..(43): n is a, c, g, or t
  misc_feature (45)..(46): n is a, c, g, or t
  misc_feature (48)..(49): n is a, c, g, or t
ccgctgtgtattactgcgccargnnknnknnknnknnknnknnknnknnkgstwtkctggacaactggggcca

SEQ ID NO: 60 - DNA, 79 bp, Artificial Sequence
  Synthesized
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
  misc_feature (36)..(37): n is a, c, g, or t
  misc_feature (39)..(40): n is a, c, g, or t
  misc_feature (42)..(43): n is a, c, g, or t
  misc_feature (45)..(46): n is a, c, g, or t
  misc_feature (48)..(49): n is a, c, g, or t
  misc_feature (51)..(52): n is a, c, g, or t
  misc_feature (54)..(55): n is a, c, g, or t
ccgctgtgtattactgcgccargnnknnknnknnknnknnknnknnknnknnknnkgstwtkctggacaactggggcca

SEQ ID NO: 61 - DNA, 55 bp, Artificial Sequence
  Synthesized
  misc_feature (21)..(22): n is a, c, g, or t
  misc_feature (24)..(25): n is a, c, g, or t
  misc_feature (27)..(28): n is a, c, g, or t
  misc_feature (30)..(31): n is a, c, g, or t
ccacctactactgccagcagnnknnknnknnkcygwtcactttcggccagggcac

SEQ ID NO: 62 - DNA, 58 bp, Artificial Sequence
  Synthesized
  misc_feature (21)..(22): n is a, c, g, or t
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
ccacctactactgccagcagnnknnknnknnknnkcygwtcactttcggccagggcac

SEQ ID NO: 63 - DNA, 70 bp, Artificial Sequence
  Synthesized
  misc_feature (21)..(22): n is a, c, g, or t
  misc_feature (24)..(25): n is a, c, g, or t
  misc_feature (27)..(28): n is a, c, g, or t
  misc_feature (30)..(31): n is a, c, g, or t
  misc_feature (33)..(34): n is a, c, g, or t
  misc_feature (36)..(37): n is a, c, g, or t
  misc_feature (39)..(40): n is a, c, g, or t
  misc_feature (42)..(43): n is a, c, g, or t
  misc_feature (45)..(46): n is a, c, g, or t
ccacctactactgccagcagnnknnknnknnknnknnknnknnknnkcygwtcactttcggccagggcac

SEQ ID NO: 64 - DNA, 27 bp, Artificial Sequence
  Synthesized
ctaccgtggcccaggacatccagatga

SEQ ID NO: 65 - DNA, 27 bp, Artificial Sequence
  Synthesized
tggtgctggccgggctggacacggtca

(19) United States
(12) Patent Application Publication
(10) Pub. No.: US 2021/0246220 A1
(43) Pub. Date: Aug. 12, 2021

(54) Anti-BCMA single-chain antibody scFv and preparation method and application thereof

(71) Applicant: BGI SHENZHEN, Shenzhen, Guangdong (CN)

(72) Inventors: Meiniang WANG, Shenzhen, Guangdong (CN); Bingjie OUYANG, Shenzhen, Guangdong (CN); Naibo YANG, Shenzhen, Guangdong (CN); Lin JIANG, Shenzhen, Guangdong (CN); Bingzhao REN, Shenzhen, Guangdong (CN); Yang LIU, Shenzhen, Guangdong (CN); Zhengqi ZHAO, Shenzhen, Guangdong (CN); Bo LI, Shenzhen, Guangdong (CN); Yong HOU, Shenzhen, Guangdong (CN); Fei WANG, Shenzhen, Guangdong (CN); Yuping GE, Shenzhen, Guangdong (CN); Xuan DONG, Shenzhen, Guangdong (CN)

(21) Appl. No.: 17/283,976

(22) PCT Filed: Oct. 10, 2018
(86) PCT No.: PCT/CN2018/109564

§ 371 Date: Apr. 9, 2021

(51) Int. Cl. C07K 16/28; G01N 33/574

(52) U.S. Cl. CPC C07K 16/2878; G01N 2333/70578; C07K 2317/622; G01N 33/57407

(57) ABSTRACT

Provided are an anti-BCMA single-chain antibody scFv and a preparation method and application thereof. The preparation method is capable of artificially synthesizing a BCMA-targeted phage display library by combining site-directed mutagenesis and random mutagenesis technologies; and acquiring 3 new-type BCMA antigen-targeted scFv strains to serve as BCMA-targeted antibodies by combining a phage display technology.

### SEQUENCE LISTING

The present application contains a Sequence Listing which has been submitted electronically in ASCII format and is hereby incorporated by reference in its entirety. Said ASCII copy is named PN151334_Sequence_listing.txt and is 30.3 kilobytes in size, and is identical to the sequence listing filed in the corresponding international application No. PCT/CN2018/109564 filed on Oct. 10, 2018, except that the “Synthesized” source of the artificial sequence of SEQ ID NO:55-58 has been added, So no new matter is introduced.

### TECHNICAL FIELD

The disclosure relates to the technical field of antibodies, and in particular to an anti-BCMA single-chain antibody scFv and a preparation method and application thereof.

### BACKGROUND

In the past few years, the adoptive T cell immunotherapy has become one of the most promising strategies in cancer immunotherapy. CAR-T therapy has attracted a great attention of researchers since the birth thereof, especially in the treatment of patients with advanced leukemia in 2011, has achieved a great success. After remission of about 80% of lymphomas is achieved in 2012, it is rapidly developed. The current CAR-T therapy is mainly based on the second-generation CAR. The treatment mode is to isolate T cells from the patient and use engineered methods to equip the T cells with genetically modified chimeric antigen receptor (CAR). The CAR is a composite membrane receptor molecule, includes extracellular and intracellular two functional parts, and has functions of specifically positioning to a target molecule and activating the T cells. The extracellular part of the CAR is a recombinant receptor (scFv) formed by antibody single-chain variable fragments. It is mediated by the scFv, and specifically targeted to the target molecule in a mode of antigen-antibody specific binding, the T cells are activated, and thereby a tumor-killing effect is achieved.

After the successful treatment of blood diseases, people began to turn their attention to the field of solid tumors, including multiple myeloma, breast cancer, prostate cancer, ovarian cancer, glioma, colorectal cancer, liver metastasis, mesothelioma, kidney cancer, sarcoma, nerve cell carcinoma and pleural mesothelioma, etc. The year of 2017 was called the first year of cell therapy, French biotechnology company Cellectis announced in March that a universal CAR-T therapy UCART123 researched and developed by itself had been approved by the United States FDA for the phase-I clinical trial of an Investigative New Drug (IND), which was the first CAR-T clinical trial approved by the FDA in the world for an allogeneic method targeting CD123; and in August of the same year, the United States FDA approved the CD19-targeted CAR-T product Kymriah of Novartis; in October, Yescarta of Kite, a product used for the treatment of adult patients with specific-type large B-cell tumors, was launched, these achievements have greatly promoted the researchers' enthusiasm for the development of the cellular immunotherapy. As the latest technology of immunotherapy, CAR-T has a wide range of applications and broad prospects. The market value of CAR-T in China is expected to exceed 100 billion.

B Cell Maturation Antigen (BCMA, CD269) is a member of the tumor necrosis factor receptor (TNFR) superfamily, and is a non-glycosylated type-III integral membrane protein composed of 185 amino acids. The BCMA binds the B cell activating factor (BAFF, also known as BLys, THANK, TALL-1) and a proliferation-inducing ligand (APRIL), and plays a vital role in processes of managing maturation of B cells and differentiating the B cells into plasma cells. It is indicated from researches that overexpression of the BCMA may cause generation of a protein kinase B, MAPK and N F-κB signals, thereby proliferation and survival of myeloma cells are enhanced. The BCMA is first found on the surface of mature B lymphocytes, and it is almost not expressed in other tissue cells, while it is highly expressed in malignant-proliferated B lymphocytes (such as myeloma cells and leukemia cells) and mediates a downstream signaling pathway. It has a critical effect on cell survival, proliferation, metastasis and drug resistance, these characteristics make it become a new drug target for diagnosis and immunotherapy of Multiple Myeloma (MM).

With the increasing incidence of MM and other B-cell-related diseases, a specific therapy for the B cells is expected to be developed clinically in a short period of time. In recent years, new tumor immunotherapy methods for the BCMA have become mature day by day, and mainly include a Chimeric Antigen Receptor T-Cell Immunotherapy (CAR-T), a bispecific antibody (BsAb) and an antibody-drug coupling (ADC). At an annual meeting of the American Association for Cancer Research (AACR) in April 2017, the BCMA becomes a star target, domestic and foreign companies rush to develop a BCMA-targeted CAR-T therapy. At present, 4 types of drugs have entered a clinical phase-I, and mainly include KITE-585, bb2121, LCAR-B38, etc. A domestic clinical application of the LCAR-B38 was submitted by Nanjing Legend Biotech Company in December 2017. It was the first domestic BCMA-CART to submit the clinical application. Data of a clinical trial involving 35 patients with relapsed or drug-resistant multiple myeloma showed that an objective remission rate of this therapy reached 100%, and 14 of the 19 patients reached the diagnostic criteria for a complete response during the early 4-month median follow-up period. At present, the research of the CAR-T therapy for the MM targeting to the BCMA is in an emerging stage. However, according to the results of pre-clinical and clinical phase-I trials, targeted drugs for the BCMA may be effectively targeted to tumor cells, killing the tumor cells through a cytotoxic effect of immune cells or small molecules, with significant drug effects and controllable adverse reactions, and it is a new target for the tumor targeted therapy with a great development potential.

With the spread of the multiple myeloma and other B cell-related diseases, the medical community urgently needs to develop a specific therapy for the B cells. However, technologies for the development of BCMA antibody drugs in the prior art are still focused on the use of a mouse-derived traditional antibody technology, and the traditional antibodies, whether large-scale expression or humanization, is more difficult, time-consuming, costly and low acquisition rate of effective antibodies, which severely limits the development of effective targeted drugs.

### SUMMARY

The disclosure provides an anti-BCMA single-chain antibody scFv and a preparation method and application thereof.

According to a first aspect, an embodiment provides an anti-BCMA single-chain antibody scFv, including a light-chain variable region (VL) and a heavy-chain variable region (VH), the above light-chain variable region (VL) includes a framework region (FR) and a complementarity determining region (CDR), herein the complementarity determining region (CDR) includes a CDR1, herein a sequence thereof is QDISNY (SEQ ID NO:14), a CDR2, herein a sequence thereof is YTS (SEQ ID NO:16), and a CDR3, herein a sequence thereof is QQYRKLAWT (SEQ ID NO:18); and the above heavy-chain variable region (VH) includes a framework region (FR) and a complementarity determining region (CDR), herein the complementarity determining region (CDR) includes a CDR1, herein a sequence thereof is GGTFSNYW (SEQ ID NO:21), a CDR2, herein a sequence thereof is TYRGHSDT (SEQ ID NO:23), and a CDR3, herein a sequence thereof is ARGAIYNGYDVLDN (SEQ ID NO:25); or the above light-chain variable region (VL) includes a framework region (FR) and a complementarity determining region (CDR), herein the complementarity determining region (CDR) includes a CDR1, herein a sequence thereof is QDISNY (SEQ ID NO:28), a CDR2, herein a sequence thereof is YTS (SEQ ID NO:30), and a CDR3, herein a sequence thereof is QQALVVTPFT (SEQ ID NO:32); and the above heavy-chain variable region (VH) includes a framework region (FR) and a complementarity determining region (CDR), herein the complementarity determining region (CDR) includes a CDR1, herein a sequence thereof is GGTFSNYW (SEQ ID NO:35), a CDR2, herein a sequence thereof is TYRGHSDT (SEQ ID NO:37), and a CDR3, herein a sequence thereof is ARFGMLDN (SEQ ID NO:39); or the above light-chain variable region (VL) includes a framework region (FR) and a complementarity determining region (CDR), herein the complementarity determining region (CDR) includes a CDR1, herein a sequence thereof is QDISNY (SEQ ID NO:42), a CDR2, herein a sequence thereof is YTS (SEQ ID NO:44), and a CDR3, herein a sequence thereof is QQRLTPSPFT(SEQ ID NO:46); and the above heavy-chain variable region (VH) includes a framework region (FR) and a complementarity determining region (CDR), herein the complementarity determining region (CDR) includes a CDR1, herein a sequence thereof is GGTFSNYW (SEQ ID NO:49), a CDR2, herein a sequence thereof is TYRGHSDT (SEQ ID NO:51), and a CDR3, herein a sequence thereof is ARNTALLDN (SEQ ID NO:53).

Preferably, the above light-chain variable region (VL) is:

(SEQ ID NO: 4)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLAWTFGQGT
KLEIKR;

the above heavy-chain variable region (VH) is:

(SEQ ID NO: 3)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARGSIF
NGYDVLDNWGQGTLVTVSS;

or

the above light-chain variable region (VL) is:

(SEQ ID NO: 8)
DIQMTQSPSSLSASVGDRVTITCSASQDISNYLNWYQQKPGKAPKLLIYYT
SNLHSGVPSRFSGSGSGTDFTLTISSLQPEDFATYYCQQALVVTPFTFGQG
TKLEIKR;

the above heavy-chain variable region (VH) is:

(SEQ ID NO: 7)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARFGML
DNWGQGTLVTVSS;

or

the above light-chain variable region (VL) is:

(SEQ ID NO: 12)
DIQMTQSPSSLSASVGDRVTITCSASQDISNYLNWYQQKPGKAPKLLIYYT
SNLHSGVPSRFSGSGSGTDFTLTISSLQPEDFATYYCQQRLTPSPFTFGQG
TKLEIK;

the above heavy-chain variable region (VH) is:

(SEQ ID NO: 11)
QVQLVQSGAEVKKPGSSVKVSCKASGGTFSNYWMHWVRQAPGQGLEWMGAT
YRGHSDTYYNQKFKGRVTITADKSTSTAYMELSSLRSEDTAVYYCARNTAL
LDNWGQGTLVTVSS.

Preferably, a sequence of the above single-chain antibody scFv is:

(SEQ ID NO: 1)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLAWTFGQGT
KLEIKRGGGGSGGGGSGGGGSGGGGSQVQLVQSGAEVKKPGSSVKVSCKAS
GGTFSNYWMHWVRQAPGQGLEWMGATYRGHSDTYYNQKFKGRVTITADKST
STAYMELSSLRSEDTAVYYCARGSIFNGYDVLDNWGQGTLVTVSS;
or
(SEQ ID NO: 5)
DIQMTQSPSSLSASVGDRVTITCSASQDISNYLNWYQQKPGKAPKLLIYYT
SNLHSGVPSRFSGSGSGTDFTLTISSLQPEDFATYYCQQALVVTPFTFGQG
TKLEIKRGGGGSGGGGSGGGGSGGGGSQVQLVQSGAEVKKPGSSVKVSCKA
SGGTFSNYWMHWVRQAPGQGLEWMGATYRGHSDTYYNQKFKGRVTITADKS
TSTAYMELSSLRSEDTAVYYCARFGMLDNWGQGTLVTVSS;
or
(SEQ ID NO: 9)
DIQMTQSPSSLSASVGDRVTITCSASQDISNYLNWYQQKPGKAPKLLIYYT
SNLHSGVPSRFSGSGSGTDFTLTISSLQPEDFATYYCQQRLTPSPFTFGQG
TKLEIKRGGGGSGGGGSGGGGSGGGGSQVQLVQSGAEVKKPGSSVKVSCKA
SGGTFSNYWMHWVRQAPGQGLEWMGATYRGHSDTYYNQKFKGRVTITADKS
TSTAYMELSSLRSEDTAVYYCARNTALLDNWGQGTLVTVSS.

According to a second aspect, an embodiment provides a polynucleotide sequence for encoding the anti-BCMA single-chain antibody scFv in the first aspect, including a nucleotide sequence for encoding complementarity determining region sequences SEQ ID NO: 14, SEQ ID NO: 16 and SEQ ID NO: 18 of the above light-chain variable region (VL); and a nucleotide sequence for encoding complementarity determining region sequences SEQ ID NO: 21, SEQ ID NO: 23, and SEQ ID NO: 25 of the above heavy-chain variable region (VH); or

the above polynucleotide sequence includes a nucleotide sequence for encoding complementarity determining region sequences SEQ ID NO: 28, SEQ ID NO: 30 and SEQ ID NO: 32 of the above light-chain variable region (VL); and a nucleotide sequence for encoding complementarity determining region sequences SEQ ID NO: 35, SEQ ID NO: 37, and SEQ ID NO: 39 of the above heavy-chain variable region (VH); or

the above polynucleotide sequence includes a nucleotide sequence for encoding complementarity determining region sequences SEQ ID NO: 42, SEQ ID NO: 44 and SEQ ID NO: 46 of the above light-chain variable region (VL); and a nucleotide sequence for encoding complementarity determining region sequences SEQ ID NO: 49, SEQ ID NO: 51, and SEQ ID NO: 53 of the above heavy-chain variable region (VH).

Preferably, the above polynucleotide sequence includes a nucleotide sequence for encoding a sequence SEQ ID NO:4 of the above light-chain variable region (VL); and a nucleotide sequence for encoding a sequence SEQ ID NO:3 of the above heavy-chain variable region (VH); or

the above polynucleotide sequence includes a nucleotide sequence for encoding a sequence SEQ ID NO:8 of the above light-chain variable region (VL); and a nucleotide sequence for encoding a sequence SEQ ID NO:7 of the above heavy-chain variable region (VH); or

the above polynucleotide sequence includes a nucleotide sequence for encoding a sequence SEQ ID NO:12 of the above light-chain variable region (VL); and a nucleotide sequence for encoding a sequence SEQ ID NO:11 of the above heavy-chain variable region (VH).

Preferably, the above polynucleotide sequence is:

(SEQ ID NO: 2)
GACATCCAGATGACCCAGAGCCCTAGCTCACTGAGCGCCAGCGTGGGCGAC
AGGGTGACCATTACCTGCTCCGCCAGCCAGGACATCAGCAACTACCTGAAC
TGGTACCAGCAGAAGCCCGGCAAGGCCCCCAAGCTGCTGATCTACTACACC
TCCAACCTGCACTCCGGCGTGCCCAGCAGGTTCAGCGGAAGCGGCAGCGGC
ACCGATTTCACCCTGACCATCTCCAGCCTGCAGCCCGAGGACTTCGCCACC
TACTACTGCCAGCAGTACAGGAAGCTCGCATGGACTTTCGGCCAGGGCACC
AAACTGGAGATCAAGCGTGGTGGAGGAGGTAGCGGAGGAGGCGGGAGCGGT
GGAGGTGGCTCTGGAGGTGGCGGAAGCCAGGTGCAGCTGGTCCAGAGCGGC
GCCGAAGTGAAGAAGCCCGGCAGCTCCGTGAAAGTGAGCTGCAAGGCCAGC
GGCGGCACCTTCAGCAACTACTGGATGCACTGGGTGAGGCAGGCCCCCGGA
CAGGGCCTGGAGTGGATGGGCGCCACCTACAGGGGCCACAGCGACACCTAC
TACAACCAGAAGTTCAAGGGCCGGGTGACCATCACCGCCGACAAGAGCACC
AGCACCGCCTACATGGAACTGAGCAGCCTCAGGAGCGAGGACACCGCTGTG
TATTACTGCGCCAGGGGTTCTATTTTCAACGGTTACGACGTTCTGGACAAC
TGGGGCCAGGGCACACTAGTGACCGTGTCCAGC;
or
(SEQ ID NO: 6)
GACATCCAGATGACCCAGAGCCCTAGCTCACTGAGCGCCAGCGTGGGCGAC
AGGGTGACCATTACCTGCTCCGCCAGCCAGGACATCAGCAACTACCTGAAC
TGGTACCAGCAGAAGCCCGGCAAGGCCCCCAAGCTGCTGATCTACTACACC
TCCAACCTGCACTCCGGCGTGCCCAGCAGGTTCAGCGGAAGCGGCAGCGGC
ACCGATTTCACCCTGACCATCTCCAGCCTGCAGCCCGAGGACTTCGCCACC
TACTACTGCCAGCAGGCTCTTGTGGTGACGCCGTTCACTTTCGGCCAGGGC
ACCAAACTGGAGATCAAGCGTGGTGGAGGAGGTAGCGGAGGAGGCGGGAGC
GGTGGAGGTGGCTCTGGAGGTGGCGGAAGCCAGGTGCAGCTGGTCCAGAGC
GGCGCCGAAGTGAAGAAGCCCGGCAGCTCCGTGAAAGTGAGCTGCAAGGCC
AGCGGCGGCACCTTCAGCAACTACTGGATGCACTGGGTGAGGCAGGCCCCC
GGACAGGGCCTGGAGTGGATGGGCGCCACCTACAGGGGCCACAGCGACACC
TACTACAACCAGAAGTTCAAGGGCCGGGTGACCATCACCGCCGACAAGAGC
ACCAGCACCGCCTACATGGAACTGAGCAGCCTCAGGAGCGAGGACACCGCT
GTGTATTACTGCGCCAGGTTTTAGGGTATGCTGGACAACTGGGGCCAGGGC
ACACTAGTGACCGTGTCCAGC;
or
(SEQ ID NO: 10)
GACATCCAGATGACCCAGAGCCCTAGCTCACTGAGCGCCAGCGTGGGCGAC
AGGGTGACCATTACCTGCTCCGCCAGCCAGGACATCAGCAACTACCTGAAC
TGGTACCAGCAGAAGCCCGGCAAGGCCCCCAAGCTGCTGATCTACTACACC
TCCAACCTGCACTCCGGCGTGCCCAGCAGGTTCAGCGGAAGCGGCAGCGGC
ACCGATTTCACCCTGACCATCTCCAGCCTGCAGCCCGAGGACTTCGCCACC
TACTACTGCCAGCAGCGTTTGACGCCGTCTCCGTTCACTTTCGGCCAGGGC
ACCAAACTGGAGATCAAGCGTGGTGGAGGAGGTAGCGGAGGAGGCGGGAGC
GGTGGAGGTGGCTCTGGAGGTGGCGGAAGCCAGGTGCAGCTGGTCCAGAGC
GGCGCCGAAGTGAAGAAGCCCGGCAGCTCCGTGAAAGTGAGCTGCAAGGCC
AGCGGCGGCACCTTCAGCAACTACTGGATGCACTGGGTGAGGCAGGCCCCC
GGACAGGGCCTGGAGTGGATGGGCGCCACCTACAGGGGCCACAGCGACACC
TACTACAACCAGAAGTTCAAGGGCCGGGTGACCATCACCGCCGACAAGAGC
ACCAGCACCGCCTACATGGAACTGAGCAGCCTCAGGAGCGAGGACACCGCT
GTGTATTACTGCGCCAGGAATACGGCTTTGCTGGACAACTGGGGCCAGGGC
ACACTAGTGACCGTGTCCAGC.

According to a third aspect, an embodiment provides an expression vector, containing the polynucleotide sequence in the second aspect.

According to a fourth aspect, an embodiment provides a host cell, containing the expression vector in the third aspect, and an anti-BCMA single-chain antibody scFv may be expressed.

According to a fifth aspect, an embodiment provides a pharmaceutical composition, containing the anti-BCMA single-chain antibody scFv in the first aspect, and a pharmaceutically acceptable carrier, a diluent or a excipient.

According to a sixth aspect, an embodiment provides a method for preparing the anti-BCMA single-chain antibody scFv in the first aspect, including: transforming an expression vector containing the polynucleotide sequence in the second aspect to an expression host cell, culturing, and performing mass expression and purification of the above anti-BCMA single-chain antibody scFv.

Preferably, the above expression vector is a pComb3XSS vector, and the above host cells are Escherichia coli XL1-Blue and a TG1 strain.

According to a seventh aspect, an embodiment provides an application of the anti-BCMA single-chain antibody scFv in the first aspect in preparing a BCMA-targeted drug, or an application in immunologically detecting BCMA for a non-disease diagnosis and treatment purpose.

A BCMA-targeted phage display library is artificially synthesized in the disclosure in combination with site-directed mutagenesis and random mutagenesis technologies, and 3 new-type BCMA antigen-targeted scFv strains are obtained by combining a phage display technology, which may be served as a BCMA-targeted antibody for further research and development.

### BRIEF DESCRIPTION OF THE DRAWINGS

FIG. 1 is an electrophoresis detection result diagram of a secondary library and a random library of site-directed mutagenesis in Embodiment 1 of the disclosure.

FIG. 2 is a recombinant efficiency result diagram of a scFv fragment in a phage display library constructed by cloning PCR verification in Embodiment 1 of the disclosure, herein clones 1-36 are clones of the secondary library, and clones 37-72 are clones of the random library.

FIG. 3 is an affinity result diagram of phage library targeting BCMA after 4 rounds of affinity biopanning detected by ELISA in Embodiment 2 of the disclosure.

FIG. 4 is a BCMA-targeted scFv monoclonal PCR screening result diagram in Embodiment 2 of the disclosure, herein clones 1-36 are clones screened from the random library, clones 37-56 are clones screened from the secondary library, and the arrow represents a clone with a mutated sequence.

FIG. 5 is an affinity result diagram of a BCMA-targeted scFv candidate strain verified by the

ELISA in Embodiment 2 of the disclosure.

### DETAILED DESCRIPTION OF THE EMBODIMENTS

The disclosure is further described in detail below through specific implementation modes in combination with drawings. In the following implementation modes, many detailed descriptions are used to enable the disclosure to be better understood. However, it may be easily realized by those skilled in the art that some of features may be omitted under different circumstances, or may be replaced by other elements, materials, and methods.

In addition, features, operations, or characteristics described in the description may be combined in any suitable modes to form the various implementation modes. At the same time, each operation or action described in the methods may also be sequentially exchanged or adjusted in a mode which is apparent to those skilled in the art. Therefore, various sequences in the description and the drawings are only used for clearly describing a certain embodiment, and are not meant to be a necessary sequence, unless it is otherwise specified that a certain sequence must be followed.

In the present disclosure, based on a humanized scFv sequence of anti-BCMA, site-directed mutagenesis and random mutagenesis are performed on CDR3 regions of heavy-chain and light-chain thereof, and a phage library of anti-BCMA scFv is artificially synthesized; then combined with a phage display technology, multiple rounds of affinity panning and screening are performed on the BCMA antigen, finally sequence analysis and affinity detection are performed on the antibody monoclonal strains obtained from screening, and new anti-BCMA scFv strains have been obtained and can be used as an antibody of a BCMA-targeted drug for the development of downstream immunotherapeutic drugs.

In the present disclosure, the humanized scFv sequence is used as a backbone, and only the CDR3 regions thereof are artificially modified, which can greatly reduce the difficulty of humanization of the antibody in the later stage, and meanwhile reduce the effect of the modification on the affinity of the antibody targeted to the antigen; at the same time, by combining the use of the phage display technology, antibody affinity information is obtained relatively quickly and intuitively and multiple new high-affinity scFv strains are obtained in a short time, which provides more ways for development of the BCMA-targeted immunotherapy, and has a value of continued development.

Based on the scFv sequence of BCMA, the CDR3 regions of the heavy-chain and light-chain thereof are mutated, and 3 new BCMA-targeted scFv antibody strains are obtained by combining with the phage display technology.

The amino acid and nucleotide sequence information of the 3 anti-BCMA scFv antibodies obtained above are as shown in Table 1:

TABLE 1
Nucleotide and amino acid sequence information of anti-BCMA
positive clones scFv_20, scFv_43 and scFv_46

| | |
|---|---|
| Name/Description | Sequence |

scFv_20

| | |
|---|---|
| scFv_20-aa scFv | DIQMTQSPSSLSASVGDRVTITCSASQDISNYLNWYQQKPGKAPKLLIYYTSNLHS |
| domain (domain) | GVPSRFSGSGSGTDFTLTISSLQPEDFATYYCQQYRKLAWTFGQGTKLEIKRGGG |
| | GSGGGGSGGGGSGGGGSQVQLVQSGAEVKKPGSSVKVSCKASGGTFSNYWM |
| | HWVRQAPGQGLEWMGATYRGHSDTYYNQKFKGRVTITADKSTSTAYMELSSLRS |
| | EDTAVYYCARGSIFNGYDVLDNWGQGTLVTVSS (SEQ ID NO: 1) |
| scFv_20-nt scFv | GACATCCAGATGACCCAGAGCCCTAGCTCACTGAGCGCCAGCGTGGGCGACA |
| domain (domain) | GGGTGACCATTACCTGCTCCGCCAGCCAGGACATCAGCAACTACCTGAACTG |
| | GTACCAGCAGAAGCCCGGCAAGGCCCCCAAGCTGCTGATCTACTACACCTCC |
| | AACCTGCACTCCGGCGTGCCCAGCAGGTTCAGCGGAAGCGGCAGCGGCACC |
| | GATTTCACCCTGACCATCTCCAGCCTGCAGCCCGAGGACTTCGCCACCTACTA |
| | CTGCCAGCAGTACAGGAAGCTCGCATGGACTTTCGGCCAGGGCACCAAACTG |
| | GAGATCAAGCGTGGTGGAGGAGGTAGCGGAGGAGGCGGGAGCGGTGGAGGT |
| | GGCTCTGGAGGTGGCGGAAGCCAGGTGCAGCTGGTCCAGAGCGGCGCCGAA |
| | GTGAAGAAGCCCGGCAGCTCCGTGAAAGTGAGCTGCAAGGCCAGCGGCGGC |
| | ACCTTCAGCAACTACTGGATGCACTGGGTGAGGCAGGCCCCCGGACAGGGCC |
| | TGGAGTGGATGGGCGCCACCTACAGGGGCCACAGCGACACCTACTACAACCA |
| | GAAGTTCAAGGGCCGGGTGACCATCACCGCCGACAAGAGCACCAGCACCGCC |
| | TACATGGAACTGAGCAGCCTCAGGAGCGAGGACACCGCTGTGTATTACTGCG |
| | CCAGGGGTTCTATTTTCAACGGTTACGACGTTCTGGACAACTGGGGCCAGGG |
| | CACACTAGTGACCGTGTCCAGC (SEQ ID NO: 2) |
| scFv_20-aa VH | QVQLVQSGAEVKKPGSSVKVSCKASGGTFSNYWMHWVRQAPGQGLEWMGATY |
| (heavy-chain | RGHSDTYYNQKFKGRVTITADKSTSTAYMELSSLRSEDTAVYYCARGSIFNGYDVL |
| variable region) | DNWGQGTLVTVSS (SEQ ID NO: 3) |
| scFv_20-aa VL | DIQMTQSPSSLSASVGDRVTITCSASQDISNYLNWYQQKPGKAPKLLIYYTSNLHS |
| (light-chain variable | GVPSRFSGSGSGTDFTLTISSLQPEDFATYYCQQYRKLAWTFGQGTKLEIKR |
| region) | (SEQ ID NO: 4) |

scFv_43

| | |
|---|---|
| scF_v43-aa scFv | DIQMTQSPSSLSASVGDRVTITCSASQDISNYLNWYQQKPGKAPKLLIYYTSNLHS |
| domain (domain) | GVPSRFSGSGSGTDFTLTISSLQPEDFATYYCQQALVVTPFTFGQGTKLEIKRGGG |
| | GSGGGGSGGGGSGGGGSQVQLVQSGAEVKKPGSSVKVSCKASGGTFSNYWM |
| | HWVRQAPGQGLEWMGATYRGHSDTYYNQKFKGRVTITADKSTSTAYMELSSLRS |
| | EDTAVYYCARFGMLDNWGQGTLVTVSS (SEQ ID NO: 5) |
| scFv_43-nt scFv | GACATCCAGATGACCCAGAGCCCTAGCTCACTGAGCGCCAGCGTGGGCGACA |
| domain (domain) | GGGTGACCATTACCTGCTCCGCCAGCCAGGACATCAGCAACTACCTGAACTG |
| | GTACCAGCAGAAGCCCGGCAAGGCCCCCAAGCTGCTGATCTACTACACCTCC |
| | AACCTGCACTCCGGCGTGCCCAGCAGGTTCAGCGGAAGCGGCAGCGGCACC |
| | GATTTCACCCTGACCATCTCCAGCCTGCAGCCCGAGGACTTCGCCACCTACTA |
| | CTGCCAGCAGGCTCTTGTGGTGACGCCGTTCACTTTCGGCCAGGGCACCAAA |
| | CTGGAGATCAAGCGTGGTGGAGGAGGTAGCGGAGGAGGCGGGAGCGGTGGA |
| | GGTGGCTCTGGAGGTGGCGGAAGCCAGGTGCAGCTGGTCCAGAGCGGCGCC |
| | GAAGTGAAGAAGCCCGGCAGCTCCGTGAAAGTGAGCTGCAAGGCCAGCGGC |
| | GGCACCTTCAGCAACTACTGGATGCACTGGGTGAGGCAGGCCCCCGGACAGG |
| | GCCTGGAGTGGATGGGCGCCACCTACAGGGGCCACAGCGACACCTACTACAA |
| | CCAGAAGTTCAAGGGCCGGGTGACCATCACCGCCGACAAGAGCACCAGCACC |
| | GCCTACATGGAACTGAGCAGCCTCAGGAGCGAGGACACCGCTGTGTATTACT |
| | GCGCCAGGTTTTAGGGTATGCTGGACAACTGGGGCCAGGGCACACTAGTGAC |
| | CGTGTCCAGC (SEQ ID NO: 6) |
| scFv_43-aa VH | QVQLVQSGAEVKKPGSSVKVSCKASGGTFSNYWMHWVRQAPGQGLEWMGATY |
| (heavy-chain | RGHSDTYYNQKFKGRVTITADKSTSTAYMELSSLRSEDTAVYYCARFGMLDNWG |
| variable region) | QGTLVTVSS (SEQ ID NO: 7) |
| scFv_43-aa VL | DIQMTQSPSSLSASVGDRVTITCSASQDISNYLNWYQQKPGKAPKLLIYYTSNLHS |
| (light-chain variable | GVPSRFSGSGSGTDFTLTISSLQPEDFATYYCQQALVVTPFTFGQGTKLEIKR |
| region) | (SEQ ID NO: 8) |

scFv_46

| | |
|---|---|
| scFv_46-aa scFv | DIQMTQSPSSLSASVGDRVTITCSASQDISNYLNWYQQKPGKAPKLLIYYTSNLHS |
| domain (domain) | GVPSRFSGSGSGTDFTLTISSLQPEDFATYYCQQRLTPSPFTFGQGTKLEIKRGG |
| | GGSGGGGSGGGGSGGGGSQVQLVQSGAEVKKPGSSVKVSCKASGGTFSNYW |
| | MHWVRQAPGQGLEWMGATYRGHSDTYYNQKFKGRVTITADKSTSTAYMELSSL |
| | RSEDTAVYYCARNTALLDNWGQGTLVTVSS (SEQ ID NO: 9) |
| scFv_46-nt scFv | GACATCCAGATGACCCAGAGCCCTAGCTCACTGAGCGCCAGCGTGGGCGACA |
| domain (domain) | GGGTGACCATTACCTGCTCCGCCAGCCAGGACATCAGCAACTACCTGAACTG |
| | GTACCAGCAGAAGCCCGGCAAGGCCCCCAAGCTGCTGATCTACTACACCTCC |
| | AACCTGCACTCCGGCGTGCCCAGCAGGTTCAGCGGAAGCGGCAGCGGCACC |
| | GATTTCACCCTGACCATCTCCAGCCTGCAGCCCGAGGACTTCGCCACCTACTA |
| | CTGCCAGCAGCGTTTGACGCCGTCTCCGTTCACTTTCGGCCAGGGCACCAAA |
| | CTGGAGATCAAGCGTGGTGGAGGAGGTAGCGGAGGAGGCGGGAGCGGTGGA |
| | GGTGGCTCTGGAGGTGGCGGAAGCCAGGTGCAGCTGGTCCAGAGCGGCGCC |
| | GAAGTGAAGAAGCCCGGCAGCTCCGTGAAAGTGAGCTGCAAGGCCAGCGGC |
| | GGCACCTTCAGCAACTACTGGATGCACTGGGTGAGGCAGGCCCCCGGACAGG |
| | GCCTGGAGTGGATGGGCGCCACCTACAGGGGCCACAGCGACACCTACTACAA |
| | CCAGAAGTTCAAGGGCCGGGTGACCATCACCGCCGACAAGAGCACCAGCACC |
| | GCCTACATGGAACTGAGCAGCCTCAGGAGCGAGGACACCGCTGTGTATTACT |
| | GCGCCAGGAATACGGCTTTGCTGGACAACTGGGGCCAGGGCACACTAGTGAC |
| | CGTGTCCAGC (SEQ ID NO: 10) |
| scFv_46-aa VH | QVQLVQSGAEVKKPGSSVKVSCKASGGTFSNYWMHWVRQAPGQGLEWMGATY |
| (heavy-chain | RGHSDTYYNQKFKGRVTITADKSTSTAYMELSSLRSEDTAVYYCARNTALLDNWG |
| variable region) | QGTLVTVSS (SEQ ID NO: 11) |
| scFv_46-aa VL | DIQMTQSPSSLSASVGDRVTITCSASQDISNYLNWYQQKPGKAPKLLIYYTSNLHS |
| (light-chain variable | GVPSRFSGSGSGTDFTLTISSLQPEDFATYYCQQRLTPSPFTFGQGTKLEIK (SEQ |
| region) | ID NO: 12) |

Amino acid sequences of the framework regions (FR) and complementarity determining regions (CDR) of the light-chains (L chain) and the heavy-chains (H chain) of the 3 scFv strains obtained above are as shown in Table 2:

TABLE 2
Amino acid sequence information of the framework regions (FR1-FR4) and
complementarity determining regions (CDR1-CDR3) of the light-chains and
heavy-chains of the anti-BCMA positive clones scFv_20, scFv_43 and scFv_23

| Clone | | | | | | | | |
|---|---|---|---|---|---|---|---|---|
| strain | Chain | FR1 | CDR1 | FR2 | CDR2 | FR3 | CDR3 | FR4 |
| scFv_20 | Light- | DIQMTQS | QDISNY | LNWYQQ | YTS | NLHSGVPSR | QQYRK | FGQGTKL |
| | chain | PSSLSASV | (SEQ ID | KPGKAP | (SEQ ID | FSGSGSGTD | LAWT | EIKR (SEQ |
| | | GDRVTITC | NO: 14) | KLLIY | NO: 16) | FTLTISSLQPE | (SEQ ID | ID NO: 19) |
| | | SAS (SEQ | | (SEQ ID | | DFATYYC | NO: 18) | |
| | | ID NO: 13) | | NO: 15) | | (SEQ ID | | |
| | | | | | | NO: 17) | | |
| scFv_20 | Heavy- | QVQLVQS | GGTFS | MHWVR | TYRG | YYNQKFKGR | ARGAIY | WGQGTLV |
| | chain | GAEVKKP | NYW | QAPGQG | HSDT | VTITADKSTS | NGYDV | TVSS |
| | | GSSVKVS | (SEQ ID | LEWMGA | (SEQ ID | TAYMELSSLR | LDN | (SEQ ID |
| | | CKAS | NO: 21) | (SEQ ID | NO: 23) | SEDTAVYYC | (SEQ ID | NO: 26) |
| | | (SEQ ID | | NO: 22) | | (SEQ ID | NO: 25) | |
| | | NO: 20) | | | | NO: 24) | | |
| scFv_43 | Light- | DIQMTQS | QDISNY | LNWYQQ | YTS | NLHSGVPSR | QQALV | FGQGTKL |
| | chain | PSSLSASV | (SEQ ID | KPGKAP | (SEQ ID | FSGSGSGTD | VTPFT | EIKR (SEQ |
| | | GDRVTITC | NO: 28) | KLLIY | NO: 30) | FTLTISSLQPE | (SEQ ID | ID NO: 33) |
| | | SAS (SEQ | | (SEQ ID | | DFATYYC | NO: 32) | |
| | | ID NO: 27) | | NO: 29) | | (SEQ ID | | |
| | | | | | | NO: 31) | | |
| scFv_43 | Heavy- | QVQLVQS | GGTFS | MHWVR | TYRG | YYNQKFKGR | ARFGM | WGQGTLV |
| | chain | GAEVKKP | NYW | QAPGQG | HSDT | VTITADKSTS | LDN | TVSS |
| | | GSSVKVS | (SEQ ID | LEWMGA | (SEQ ID | TAYMELSSLR | (SEQ ID | (SEQ ID |
| | | CKAS | NO: 35) | (SEQ ID | NO: 37) | SEDTAVYYC | NO: 39) | NO: 40) |
| | | (SEQ ID | | NO: 36) | | (SEQ ID | | |
| | | NO: 34) | | | | NO: 38) | | |
| scFv_46 | Light- | DIQMTQS | QDISNY | LNWYQQ | YTS | NLHSGVPSR | QQRLT | FGQGTKL |
| | chain | PSSLSASV | (SEQ ID | KPGKAP | (SEQ ID | FSGSGSGTD | PSPFT | EIKR (SEQ |
| | | GDRVTITC | NO: 42) | KLLIY | NO: 44) | FTLTISSLQPE | (SEQ ID | ID NO: 47) |
| | | SAS (SEQ | | (SEQ ID | | DFATYYC | NO: 46) | |
| | | ID NO: 41) | | NO: 43) | | (SEQ ID | | |
| | | | | | | NO: 45) | | |
| scFv_46 | Heavy- | QVQLVQS | GGTFS | MHWVR | TYRG | YYNQKFKGR | ARNTA | WGQGTLV |
| | chain | GAEVKKP | NYW | QAPGQG | HSDT | VTITADKSTS | LLDN | TVSS |
| | | GSSVKVS | (SEQ ID | LEWMGA | (SEQ ID | TAYMELSSLR | (SEQ ID | (SEQ ID |
| | | CKAS | NO: 49) | (SEQ ID | NO: 51) | SEDTAVYYC | NO: 53) | NO: 54) |
| | | (SEQ ID | | NO: 50) | | (SEQ ID | | |
| | | NO: 48) | | | | NO: 52) | | |

The scFv antibody obtained in the disclosure may be efficiently targeted and bound to the BCMA antigen and is applied to a purpose of BCMA-targeted diagnosis or treatment.

It should be to be noted that it is well-known that specific binding properties of the antibodies are determined by the complementarity determining regions. Therefore, a technical scheme of the disclosure provides an anti-BCMA single-chain antibody scFv, including a light-chain variable region (VL) and a heavy-chain variable region (VH), the light-chain variable region (VL) includes a framework region (FR) and a complementarity determining region (CDR), herein the complementarity determining region (CDR) includes a CDR1, herein a sequence thereof is QDISNY (SEQ ID NO:14), a CDR2, herein a sequence thereof is YTS (SEQ ID NO:16), and a CDR3, herein a sequence thereof is QQYRKLAWT (SEQ ID NO:18); and the heavy-chain variable region (VH) includes a framework region (FR) and a complementarity determining region (CDR), herein the complementarity determining region (CDR) includes a CDR1, herein a sequence thereof is GGTFSNYW (SEQ ID NO:21), a CDR2, herein a sequence thereof is TYRGHSDT (SEQ ID NO:23), and a CDR3, herein a sequence thereof is ARGAIYNGYDVLDN (SEQ ID NO:25); or the light-chain variable region (VL) includes a framework region (FR) and a complementarity determining region (CDR), herein the complementarity determining region (CDR) includes a CDR1, herein a sequence thereof is QDISNY (SEQ ID NO:28), a CDR2, herein a sequence thereof is YTS (SEQ ID NO:30), and a CDR3, herein a sequence thereof is QQALVVTPFT (SEQ ID NO:32); and the heavy-chain variable region (VH) includes a framework region (FR) and a complementarity determining region (CDR), herein the complementarity determining region (CDR) includes a CDR1, herein a sequence thereof is GGTFSNYW (SEQ ID NO:35), a CDR2, herein a sequence thereof is TYRGHSDT (SEQ ID NO:37), and a CDR3, herein a sequence thereof is ARFGMLDN (SEQ ID NO:39); or the light-chain variable region (VL) includes a framework region (FR) and a complementarity determining region (CDR), herein the complementarity determining region (CDR) includes a CDR1, herein a sequence thereof is QDISNY (SEQ ID NO:42), a CDR2, herein a sequence thereof is YTS (SEQ ID NO:44), and a CDR3, herein a sequence thereof is QQRLTPSPFT (SEQ ID NO:46); and the heavy-chain variable region (VH) includes a framework region (FR) and a complementarity determining region (CDR), herein the complementarity determining region (CDR) includes a CDR1, herein a sequence thereof is GGTFSNYW (SEQ ID NO:49), a CDR2, herein a sequence thereof is TYRGHSDT (SEQ ID NO:51), and a CDR3, herein a sequence thereof is ARNTALLDN (SEQ ID NO:53).

In a preferred technical scheme, the anti-BCMA single-chain antibody scFv includes a light-chain variable region (VL) and a heavy-chain variable region (VH), herein, a sequence of the light-chain variable region (VL) is SEQ ID NO:4, and a sequence of the heavy-chain variable region (VH) is SEQ ID NO:3; or a sequence of the light-chain variable region (VL) is SEQ ID NO:8, and a sequence of the heavy-chain variable region (VH) is SEQ ID NO:7; or a sequence of the light-chain variable region (VL) is SEQ ID NO:12, and a sequence of the heavy-chain variable region (VH) is SEQ ID NO:11.

In a more preferred technical scheme, a sequence of the anti-BCMA single-chain antibody scFv is SEQ ID NO:1; or SEQ ID NO:5; or SEQ ID NO:9.

In view of the degeneracy of an encoding gene, and at the same time, it is considered that the specific binding characteristics of the antibodies are determined by the complementarity determining regions. Therefore, a technical scheme of the disclosure provides a polynucleotide sequence for encoding the anti-BCMA single-chain antibody scFv of the disclosure, including a nucleotide sequence for encoding complementarity determining region sequences SEQ ID NO: 14, SEQ ID NO: 16 and SEQ ID NO: 18 of the light-chain variable region (VL); and a nucleotide sequence for encoding complementarity determining region sequences SEQ ID NO: 21, SEQ ID NO: 23, and SEQ ID NO: 25 of the heavy-chain variable region (VH); or the polynucleotide sequence includes a nucleotide sequence for encoding complementarity determining region sequences SEQ ID NO: 28, SEQ ID NO: 30 and SEQ ID NO: 32 of the light-chain variable region (VL); and a nucleotide sequence for encoding complementarity determining region sequences SEQ ID NO: 35, SEQ ID NO: 37, and SEQ ID NO: 39 of the heavy-chain variable region (VH); or the polynucleotide sequence includes a nucleotide sequence for encoding complementarity determining region sequences SEQ ID NO: 42, SEQ ID NO: 44 and SEQ ID NO: 46 of the light-chain variable region (VL); and a nucleotide sequence for encoding complementarity determining region sequences SEQ ID NO: 49, SEQ ID NO: 51, and SEQ ID NO: 53 of the heavy-chain variable region (VH). Due to the degeneracy of the encoding gene, the base sequence of such a polynucleotide sequence may be changed, as long as the respective corresponding complementarity determining regions may be encoded.

In a preferred technical scheme, the polynucleotide sequence includes a nucleotide sequence for encoding a sequence SEQ ID NO:4 of the light-chain variable region (VL); and a nucleotide sequence for encoding a sequence SEQ ID NO:3 of the heavy-chain variable region (VH); or the polynucleotide sequence includes a nucleotide sequence for encoding a sequence SEQ ID NO:8 of the light-chain variable region (VL); and a nucleotide sequence for encoding a sequence SEQ ID NO:7 of the heavy-chain variable region (VH); or the polynucleotide sequence includes a nucleotide sequence for encoding a sequence SEQ ID NO:12 of the light-chain variable region (VL); and a nucleotide sequence for encoding a sequence SEQ ID NO:11 of the heavy-chain variable region (VH).

In a more preferred technical scheme, the polynucleotide sequence is: SEQ ID NO: 2; or SEQ ID NO: 6; or SEQ ID NO: 10.

In an embodiment of the disclosure, a phagemid expression vector is provided, including the polynucleotide sequence of the disclosure. It is known to those skilled in the art that under the spirit of the disclosure, many vectors, such as pComb3XSS, pComb3XTT, and pComb3HSS, may be used as the expression vectors of the polynucleotide sequence of the disclosure. In a preferred embodiment, the expression vector is a phage display vector pComb3XSS (purchased from Wuhan Miaoling Biotechnology Co., Ltd).

In an embodiment of the disclosure, a host cell is provided, containing the expression vector of the disclosure, capable of expressing an anti-BCMA single-chain antibody scFv on the surface of a phage. It is known to those skilled in the art that under the spirit of the disclosure, many cells such as ER2738, SS320, TG1, XL1-Blue, ect. may be used as the host cells for the expression vectors of the disclosure. In a preferred embodiment, the host cells are an Escherichia coli XL1-Blue strain (purchased from TAKARA) and a TG1 strain (purchased from Lucigen, USA).

In an embodiment of the disclosure, a pharmaceutical composition is provided, containing the anti-BCMA single-chain antibody scFv of the disclosure, and a pharmaceutically acceptable carrier, a diluent or an excipient.

The pharmaceutical composition of the disclosure may be prepared by methods well-known in the field (for example, Remington: The Science and Practice of Pharmacy, 19th ed. (1995), A. Gennaro et al., Mack Publishing Co.), and includes the anti-BCMA single-chain antibody scFv disclosed in the disclosure and one or more pharmaceutically acceptable carriers, diluents or excipients.

In an embodiment of the disclosure, a method for preparing the anti-BCMA single-chain antibody scFv of the disclosure is provided, including transforming an expression vector containing the polynucleotide sequence of the disclosure to an expression host cell, culturing, and performing mass expression and purification of the anti-BCMA single-chain antibody scFv. In a preferred embodiment, the expression vector is a phage display vector pComb3XSS, and the host cells are an Escherichia coli XL1-Blue strain and a TG1 strain.

The anti-BCMA single-chain antibody scFv of the disclosure may be used to prepare anti-BCMA protein monoclonal antibody drugs and may also be used to immunologically detect the BCMA. Therefore, in an embodiment of the disclosure, an application of the anti-BCMA single-chain antibody scFv of the disclosure in preparing a BCMA-targeted drug, or an application in immunologically detecting BCMA for a non-disease diagnosis and treatment purpose is provided.

The technical schemes of the disclosure are described in detail below through the embodiments, and it should be understood that the embodiments are only exemplary and may not be understood as limitation to the scope of protection of the disclosure.

### Embodiment 1: Construction of Phage Display Library for Randomly Synthesized Single-Chain Antibodies

(1) Mutation Design of CDR3 Region

Based on heavy-chain and light-chain sequences of the humanized scFv of anti-BCMA, the mutation primers were designed respectively, and mutations were introduced in CDR3 regions of the heavy-chain and the light-chain, respectively, to construct a synthetic secondary library (for site-directed mutation) and random library (for random mutation), and mutation primer sequences are shown in Table 3.

TABLE 3
Primer information used for anti-BCMA antibody scFv mutation

| Library | Primer | Primer sequence (5′ to 3′) |
|---|---|---|
| name | name | (for Circle amplification of single primer) |
| Secondary | H3_primer | GTGTATTACTGCGCCARGGSTKCTRTTTWCRACGSTTWCGAMRT |
| library | | TCTGGACAACTGGGGC (SEQ ID NO: 55) |
| | L3_ primer | TACTACTGCCAGCAGTWCARGARGMTCSCATKGACTTTCGGCCA |
| | | GGGC (SEQ ID NO: 56) |
| Random | H3_2 | CCGCTGTGTATTACTGCGCCARGNNKNNKGSTWTKCTGGACAAC |
| library | | TGGGGCCA (SEQ ID NO: 57) |
| | H3_4 | CCGCTGTGTATTACTGCGCCARGNNKNNKNNKNNKGSTVVTKCTG |
| | | GACAACTGGGGCCA (SEQ ID NO: 58) |
| | H3_9 | CCGCTGTGTATTACTGCGCCARGNNKNNKNNKNNKNNKNNKNNK |
| | | NNKNNKGSTVVTKCTGGACAACTGGGGCCA (SEQ ID NO: 59) |
| | H3_11 | CCGCTGTGTATTACTGCGCCARGNNKNNKNNKNNKNNKNNKNNK |
| | | NNKNNKNNKNNKGSTWTKCTGGACAACTGGGGCCA (SEQ ID |
| | | NO: 60) |
| | L3_4 | CCACCTACTACTGCCAGCAGNNKNNKNNKNNKCYGWTCACTTTC |
| | | GGCCAGGGCAC (SEQ ID NO: 61) |
| | L3_5 | CCACCTACTACTGCCAGCAGNNKNNKNNKNNKNNKCYGWTCACT |
| | | TTCGGCCAGGGCAC (SEQ ID NO: 62) |
| | L3_9 | CCACCTACTACTGCCAGCAGNNKNNKNNKNNKNNKNNKNN KNNK |
| | | NNKCYGWTCACTTTCGGCCAGGGCAC (SEQ ID NO: 63) |

(Note: merged base K = G/T, M = A/C, R = A/G, S = G/C, W = A/T, Y = C/T)

(2) Template Preparation

Heavy-chain and light-chain variable region genes of the BCMA antibody were synthesized and inserted into the pComb3XSS vector (purchased from Wuhan Miaoling Biotechnology Co., Ltd) from the site Sfi I, and the primer information for antibody fragment amplification is shown in Table 4.

TABLE 4
Primer information used for BCMA antibody
fragment amplification

| Primer name | Primer sequence (5′ to 3′) |
|---|---|
| Primer_F | CTACCGTGGCCCAGGACATCCAGATGA |
| | (SEQ ID NO: 64) |
| Primer_R | TGGTGCTGGCCGGGCTGGACACGGTCA |
| | (SEQ ID NO: 65) |

1 μL of a constructed template plasmid was taken, and XL1-Blue competent cells (purchased from TAKARA) were transformed, resuscitation was performed at 37° C. for 1 h, 200 μL was taken for coating a plate, and the coated plate was incubated at 37° C. overnight.

A single clone was picked to an LB medium containing ampicillin (Amp+) M13KO7 (helper phage) (laboratory preparation), and cultured overnight at 37° C.

Bacterial solution was centrifuged to collect the supernatant, and 115 volume of PEG/NaCl was added and mixed uniformly, it was incubated on ice for 30 min, phage particles were precipitated, centrifugation was performed at 12,000 rpm for 30 min, the supernatant was discarded, the phage particles were resuspended with PBS, and the centrifugation was performed again at 12,000 rpm for 5 min, supernatant was collected and added to cultured CJ236 bacterial solution (purchased from TAKARA), it was used for forming dU-ssDNA (uracil-containing single-stranded template DNA), it was cultured at 37° C. for 30 min, M13KO7 was added, and it was continuously cultured for 1 h, the bacterial solution was transferred to 300 ml of a medium containing Amp+, Kanamycin (Kan+) and Uridine, and it was cultured overnight at 37° C.

The bacterial solution was centrifuged, supernatant was collected, 1/4 volume of PEG/NaCl was added, and mixed uniformly, it was incubated on ice for 30 min, phage particles were precipitated, centrifugation was performed at 12000 rpm for 25 min, the supernatant was discarded, the phage particles were collected, and a DNA extraction kit was used to extract dU-ssDNA, and it was quantified.

(3) Library Construction

Dry powder of each primer was prepared into 100pM of working solution, and the random library primers were mixed into heavy-chain primers H3-211 (H3-2, H3-4, H3-9, H3-11, and each 5 μL) and light-chain primers L3-459 (L3-4, L3-5, L3-9, and each 5 μL).

Phosphorylation reactions were performed on each primer of the secondary library and the random library, 2 reactions per library, and 20 μL per reaction system, it was as follows: primer (100 pM) 2 μL, 1033 T4 Buffer 2 μL, T4 polynucleotide kinase 2 μL, and deionized water 14 μL. A water bath was performed at 37° C. for 1.5h.

Phosphorylated mutation primers corresponding to the secondary library and random library were mixed respectively and annealed with the dU-ssDNA template, 1 reaction per library, and 250 μL per reaction system, it was as follows: primers (each library includes light-chain and heavy-chain 2 primers) 40 μL, 10× T4 Buffer 25 μL, dU-ssDNA template 20 μL, and deionized water supplemented to 250 μL.

An annealing program was set in a PCR instrument: 90° C., 5 min; 75° C., 45s; 70° C., 1 min; 65° C., 1 min; 60° C., 1 min; 55° C., 5 min; 50° C., 5 min; 45° C., 30s; 37 ° C., 10 min; 30° C., 45s; 25° C., 45s; 22° C., 90 s; 20° C., 5 min; and 1 cycle.

The followings were added in each annealing reaction: 10× T4 Buffer 10 μL, dNTP (each 10 mM) 25 μL, DTT (100 mM) 5 μL, and T7 DNA polymerase 3 μL. An extension reaction was performed at 37° C. for 4 h.

5 μL of T4 ligase was added to each system, and a ligation reaction was performed for 2 h at a room temperature.

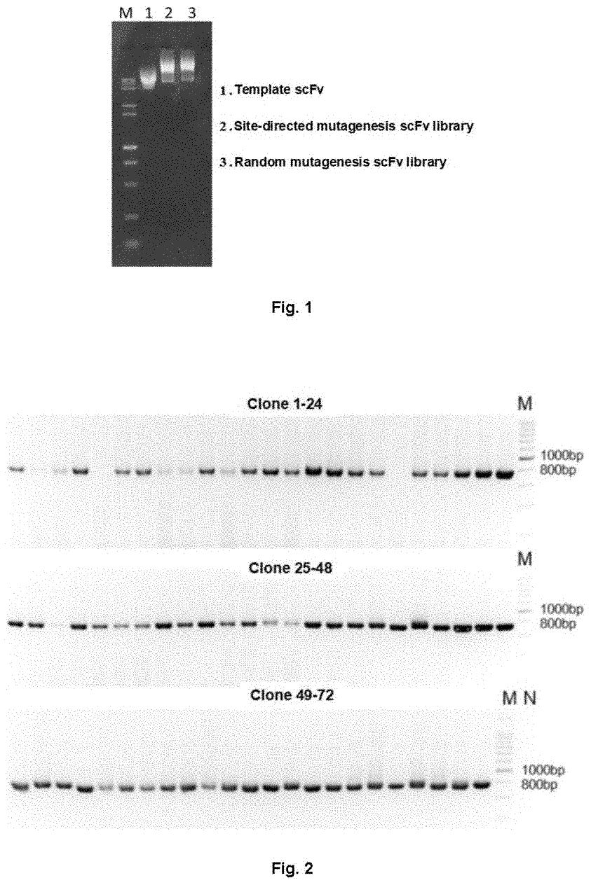

A ligation product was verified by DNA electrophoresis. As shown in FIG. 1, the band was significantly shifted compared to the template, indicating that the scFv was successfully constructed on the vector.

(4) Construction of Phage Display Library

15 μL of 3M NaAc (pH 5.5) was respectively added to the secondary library and random library to adjust a pH, the ligation product was purified by a PCR Purification Kit (QIAGEN), and a purified product was quantified with Nanodrop; 1 μL of the purified secondary library and random library were respectively taken to transform high-efficiency TG competent cells (Lucigen, USA), resuscitation was performed at 37° C. for 1 h, and it was gradient-diluted to 10⁶, 300 μL was respectively taken to coat a plate, it was cultured overnight at 37° C.; and the number of clones on the plate was counted on the next day, according to a formula:

Library capacity=number of clones*dilution*1000/30

The library capacity of the secondary library and random library obtained by calculating was greater than 10⁸/ml. All clones were eluted with LB, centrifugation was performed at 5,000 g for 5 min, and a precipitate was suspended with 2 ml of the LB, an equal volume of 30% glycerol was added, and it was frozen at −80° C.

(5) Diversity Detection of Phage Display Library

36 clones were randomly picked from the secondary library and the random library in the step (4) respectively, and served as templates to perform clone PCR, PCR products were detected through 1.5% agarose gel electrophoresis, as shown in FIG. 2, recombination rates of the secondary library (clone 1-36) and random library (clone 37-72) were 94% and 100%. 56 clones were randomly selected and sent to Sanger sequencing to analyze the diversity of the phage display library; the results of the analysis of the scFv sequence showed that 11 of the 26 sequences in the secondary library had mutations, and 14 of the 25 sequences in the random library had mutations, the diversity reached 42% and 56% respectively, it may be used for the next step of screening of new antibodies.

(6) Phage Amplification and Rescue

Proliferation and phage rescue were performed on the phage display of the secondary library and the random library obtained above, 1 ml of the phage library stored in the step (4) was respectively inoculated in 100 ml of a medium and cultured to a logarithmic growth phase, and a helper phage M13KO1 of which MOI (multiplicity of infection) was 20 was added, it was standing for 30 min at a room temperature, after low-speed centrifugation, the precipitate was suspended with a medium, inoculated in 300 ml medium, and cultured overnight. On the next day, centrifugation was performed for 30 min at 3,000, supernatant was collected, and PEG was added to precipitate the phage, it was standing on ice for 30 min, the centrifugation was performed at 3,000 for 30 min, and the precipitate was the phage library of the scFv carrying the BCMA, after the precipitate was suspended with PBS, a titer thereof was measured, the secondary library was 2.6 × 10¹² pfu/ml, and the random library was 3.3×10¹² pfu/ml.

### Embodiment 2: High-Affinity Antibodies of BCMA Screened With Phage Display Technology

(1) Phage Affinity Panning

100 ng of a BCMA-his antigen-coated ELISA plate was respectively taken as a sample well, 100 μL of Na₂CO₃ was served as a negative control well, and it was incubated overnight at 4° C. On the next day, the phage libraries of the secondary library and random library rescued in the step (6) of Embodiment 1 were added respectively, and incubated at a room temperature for 2 h; the wells were washed with PBST for 10 times, 100 μL of triethylamine was added, and incubated at a room temperature for 30 min, the collected phage was the phage library of the anti-BCMA scFv obtained by affinity panning; 10 μL of infected TG cells were taken to coat a plate, the biopanning result of the affinity phage was observed on the next day, and the remaining phages were used for amplification and rescue.

(2) Amplification and Rescue of Phage After Biopanning

Amplification and rescue methods were the same as the step (6) in Embodiment 1, the obtained PBS suspension, which was the amplified phage after the first round of biopanning, is stored at 4° C. and used for the next round of screening; and according to the biopanning step of the step (1) in Embodiment 2, an amount of the antigen was gradually decreased in each round, and the biopanning was performed for 3-4 rounds.

(3) Enrichment Degree of Specific Antibodies Evaluated by ELISA

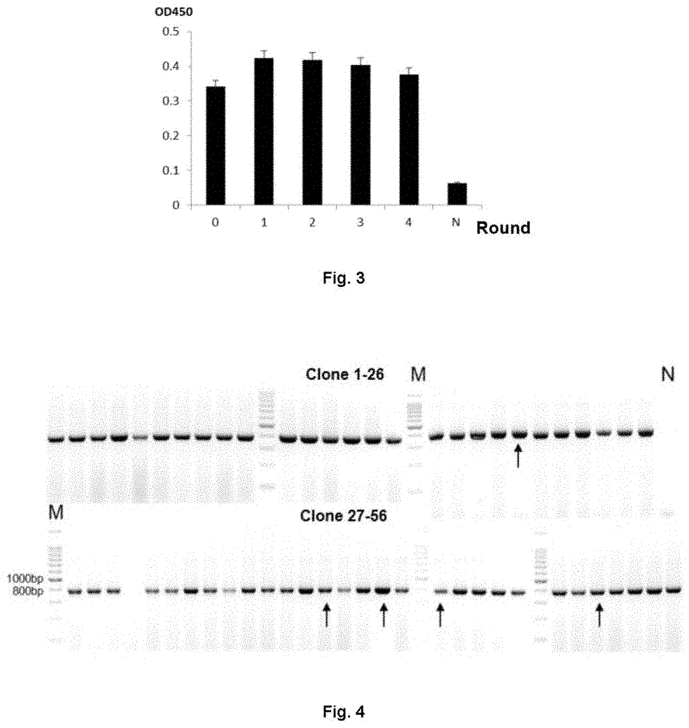

100 ng of BCMA-his antigen was taken, and placed at 4° C. overnight; on the next day, 2% BSA was added, and blocked for 1 h at a room temperature; the phage amplified after each round of the biopanning was respectively added in an experimental group, and the same amount of a wild-type phage was added in a control group, and incubated at a room temperature for 2 h; it was washed with PBST for 10 times to remove unbound phages; a HRP-labeled anti-M13 antibody was added, and incubated at a room temperature for 1 h; color developing solution was added, and reacted for 10-30 min in the dark, an absorbance value OD₄₅₀ was measured, and the ELISA result was shown in FIG. 3, the absorbance value tended to be stable from the first to the third round of the biopanning, indicating that the new specific antibodies were enriched.

(4) Identification of BCMA-Targeted High-Affinity scFv Positive Clones

The phage-coated plates obtained in the second and third rounds of biopanning were taken, and 36 and 20 clones from the random library and the secondary library were selected respectively for clone PCR; the results were shown in FIG. 4, and clones with shifted band were selected for sequencing; the sequencing results were analyzed, and it was discovered that there were 5 scFv monoclonal strains (indicated by arrows in the figure) with mutations in the CDR3 regions.

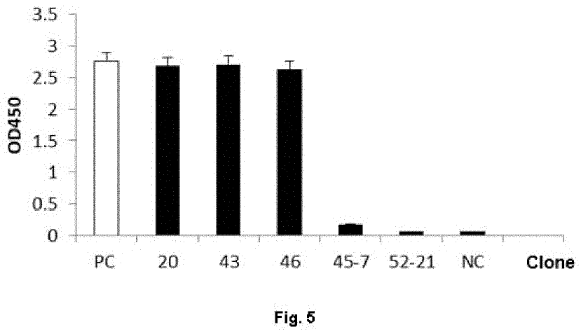

The ELISA plate was coated with 100 ng BCMA-Fc antigen, and incubated overnight at 4° C.; 5 single clones were picked and placed in 1 ml medium, cultured at 37° C. to the logarithmic phase, and induced overnight by adding 1 mM IPTG; on the next day, the bacterial precipitate was collected by centrifugation, after crushed, centrifuged at 5,000 g for 15 min, and supernatant was collected; at the same time, the ELISA plate was taken, 2% of BSA was added and blocked for 1 h at a room temperature; monoclonal crushed supernatant was added to each well of the experimental group, and blank TG crushed supernatant was added to the control group, and incubated at a room temperature for 2 h; it was washed with PBST for 10 times, a mouse anti-HA-tag antibody was added, and placed at a room temperature for 1 h; it was washed with the PBST for 3-5 times, an AP-labeled anti-mouse IgG antibody was added, and placed at a room temperature for 1 h; the substrate was added, and reacted for 10-20 min, an absorbance value was read in a microplate reader; while a ratio of the absorbance value and the control well was greater than 2.1 (baseline), it was judged to be a positive clone; and an ELISA verification result showed that 3 positive clones were obtained, namely a clone 20, a clone 43 and a clone 46 (FIG. 5).

(5) Sequence Information of Positive Clones

The 3 positive clones obtained in the step (4) were compared with the original sequence, it was discovered that they had significant mutations in the CDR3 region of the heavy-chain and the CDR3 region of the light-chain. The 3 scFv monoclonal strains were respectively named as scFv_20, scFv_43, and scFv_46, and nucleotide and amino acid sequences thereof are as follows:

scFv_20 nucleotide sequence:
(SEQ ID NO: 2)
5′-GACATCCAGATGACCCAGAGCCCTAGCTCACTGAGCGCCAGCGTGGGC
GACAGGGTGACCATTACCTGCTCCGCCAGCCAGGACATCAGCAACTACCTG
AACTGGTACCAGCAGAAGCCCGGCAAGGCCCCCAAGCTGCTGATCTACTAC
ACCTCCAACCTGCACTCCGGCGTGCCCAGCAGGTTCAGCGGAAGCGGCAGC
GGCACCGATTTCACCCTGACCATCTCCAGCCTGCAGCCCGAGGACTTCGCC
ACCTACTACTGCCAGCAGTACAGGAAGCTCGCATGGACTTTCGGCCAGGGC
ACCAAACTGGAGATCAAGCGTGGTGGAGGAGGTAGCGGAGGAGGCGGGAGC
GGTGGAGGTGGCTCTGGAGGTGGCGGAAGCCAGGTGCAGCTGGTCCAGAGC
GGCGCCGAAGTGAAGAAGCCCGGCAGCTCCGTGAAAGTGAGCTGCAAGGCC
AGCGGCGGCACCTTCAGCAACTACTGGATGCACTGGGTGAGGCAGGCCCCC
GGACAGGGCCTGGAGTGGATGGGCGCCACCTACAGGGGCCACAGCGACACC
TACTACAACCAGAAGTTCAAGGGCCGGGTGACCATCACCGCCGACAAGAGC
ACCAGCACCGCCTACATGGAACTGAGCAGCCTCAGGAGCGAGGACACCGCT
GTGTATTACTGCGCCAGGGGTTCTATTTTCAACGGTTACGACGTTCTGGAC
AACTGGGGCCAGGGCACACTAGTGACCGTGTCCAGC-3′.
scFv_20 amino acid sequence:
(SEQ ID NO: 1)
DIQMTQSPSSLSASVGDRVTITCSASQDISNYLNWYQQKPGKAPKLLIYYT
SNLHSGVPSRFSGSGSGTDFTLTISSLQPEDFATYYCQQYRKLAWTFGQGT
KLEIKRGGGGSGGGGSGGGGSGGGGSQVQLVQSGAEVKKPGSSVKVSCKAS
GGTFSNYWMHWVRQAPGQGLEWMGATYRGHSDTYYNQKFKGRVTITADKST
STAYMELSSLRSEDTAVYYCARGSIFNGYDVLDNWGQGTLVTVSS.
scFv_43 nucleotide sequence:
(SEQ ID NO: 6)
5′-GACATCCAGATGACCCAGAGCCCTAGCTCACTGAGCGCCAGCGTGGGC
GACAGGGTGACCATTACCTGCTCCGCCAGCCAGGACATCAGCAACTACCTG
AACTGGTACCAGCAGAAGCCCGGCAAGGCCCCCAAGCTGCTGATCTACTAC
ACCTCCAACCTGCACTCCGGCGTGCCCAGCAGGTTCAGCGGAAGCGGCAGC
GGCACCGATTTCACCCTGACCATCTCCAGCCTGCAGCCCGAGGACTTCGCC
ACCTACTACTGCCAGCAGGCTCTTGTGGTGACGCCGTTCACTTTCGGCCAG
GGCACCAAACTGGAGATCAAGCGTGGTGGAGGAGGTAGCGGAGGAGGCGGG
AGCGGTGGAGGTGGCTCTGGAGGTGGCGGAAGCCAGGTGCAGCTGGTCCAG
AGCGGCGCCGAAGTGAAGAAGCCCGGCAGCTCCGTGAAAGTGAGCTGCAAG
GCCAGCGGCGGCACCTTCAGCAACTACTGGATGCACTGGGTGAGGCAGGCC
CCCGGACAGGGCCTGGAGTGGATGGGCGCCACCTACAGGGGCCACAGCGAC
ACCTACTACAACCAGAAGTTCAAGGGCCGGGTGACCATCACCGCCGACAAG
AGCACCAGCACCGCCTACATGGAACTGAGCAGCCTCAGGAGCGAGGACACC
GCTGTGTATTACTGCGCCAGGTTTTAGGGTATGCTGGACAACTGGGGCCAG
GGCACACTAGTGACCGTGTCCAGC-3′.
scFv_43 amino acid sequence:
(SEQ ID NO: 5)
DIQMTQSPSSLSASVGDRVTITCSASQDISNYLNWYQQKPGKAPKLLIYYT
SNLHSGVPSRFSGSGSGTDFTLTISSLQPEDFATYYCQQALVVTPFTFGQG
TKLEIKRGGGGSGGGGSGGGGSGGGGSQVQLVQSGAEVKKPGSSVKVSCKA
SGGTFSNYWMHWVRQAPGQGLEWMGATYRGHSDTYYNQKFKGRVTITADKS
TSTAYMELSSLRSEDTAVYYCARFGMLDNWGQGTLVTVSS.
scFv_46 nucleotide sequence:
(SEQ ID NO: 10)
5′-GACATCCAGATGACCCAGAGCCCTAGCTCACTGAGCGCCAGCGTGGGC
GACAGGGTGACCATTACCTGCTCCGCCAGCCAGGACATCAGCAACTACCTG
AACTGGTACCAGCAGAAGCCCGGCAAGGCCCCCAAGCTGCTGATCTACTAC
ACCTCCAACCTGCACTCCGGCGTGCCCAGCAGGTTCAGCGGAAGCGGCAGC
GGCACCGATTTCACCCTGACCATCTCCAGCCTGCAGCCCGAGGACTTCGCC
ACCTACTACTGCCAGCAGCGTTTGACGCCGTCTCCGTTCACTTTCGGCCAG
GGCACCAAACTGGAGATCAAGCGTGGTGGAGGAGGTAGCGGAGGAGGCGGG
AGCGGTGGAGGTGGCTCTGGAGGTGGCGGAAGCCAGGTGCAGCTGGTCCAG
AGCGGCGCCGAAGTGAAGAAGCCCGGCAGCTCCGTGAAAGTGAGCTGCAAG
GCCAGCGGCGGCACCTTCAGCAACTACTGGATGCACTGGGTGAGGCAGGCC
CCCGGACAGGGCCTGGAGTGGATGGGCGCCACCTACAGGGGCCACAGCGAC
ACCTACTACAACCAGAAGTTCAAGGGCCGGGTGACCATCACCGCCGACAAG
AGCACCAGCACCGCCTACATGGAACTGAGCAGCCTCAGGAGCGAGGACACC
GCTGTGTATTACTGCGCCAGGAATACGGCTTTGCTGGACAACTGGGGCCAG
GGCACACTAGTGACCGTGTCCAGC-3′.
scFv_46 amino acid sequence:
(SEQ ID NO: 9)
DIQMTQSPSSLSASVGDRVTITCSASQDISNYLNWYQQKPGKAPKLLIYYT
SNLHSGVPSRFSGSGSGTDFTLTISSLQPEDFATYYCQQRLTPSPFTFGQG
TKLEIKRGGGGSGGGGSGGGGSGGGGSQVQLVQSGAEVKKPGSSVKVSCKA
SGGTFSNYWMHWVRQAPGQGLEWMGATYRGHSDTYYNQKFKGRVTITADKS
TSTAYMELSSLRSEDTAVYYCARNTALLDNWGQGTLVTVSS.

The disclosure is described by using specific examples above, which are only used to facilitate the understanding of the disclosure, and not used to limit the disclosure. For those skilled in the art of the disclosure, a plurality of simple deductions, modifications or replacements may also be made according to ideas of the disclosure.

What is claimed is:

1. An anti-BCMA single-chain antibody scFv, wherein the single-chain antibody scFv comprises a light-chain variable region (VL) and a heavy-chain variable region (VH),
the light-chain variable region (VL) comprises a framework region (FR) and a complementarity determining region (CDR), wherein the complementarity determining region (CDR) comprises a CDR1, wherein a sequence thereof is QDISNY (SEQ ID NO:14), a CDR2, wherein a sequence thereof is YTS (SEQ ID NO:16), and a CDR3, wherein a sequence thereof is QQYRKLAVVT (SEQ ID NO:18); and the heavy-chain variable region (VH) comprises a framework region (FR) and a complementarity determining region (CDR), wherein the complementarity determining region (CDR) comprises a CDR1, wherein a sequence thereof is GGTFSNYW (SEQ ID NO:21), a CDR2, wherein a sequence thereof is TYRGHSDT (SEQ ID NO:23), and a CDR3, wherein a sequence thereof is ARGAIYNGYDVLDN (SEQ ID NO:25); or
the light-chain variable region (VL) comprises a framework region (FR) and a complementarity determining region (CDR), wherein the complementarity determining region (CDR) comprises a CDR1, wherein a sequence thereof is QDISNY (SEQ ID NO:28), a CDR2, wherein a sequence thereof is YTS (SEQ ID NO:30), and a CDR3, wherein a sequence thereof is QQALVVTPFT (SEQ ID NO:32); and the heavy-chain variable region (VH) comprises a framework region (FR) and a complementarity determining region (CDR), wherein the complementarity determining region (CDR) comprises a CDR1, wherein a sequence thereof is GGTFSNYW (SEQ ID NO:35), a CDR2, wherein a sequence thereof is TYRGHSDT (SEQ ID NO:37), and a CDR3, wherein a sequence thereof is ARGAIYNGYDVLDN (SEQ ID NO:39); or
the light-chain variable region (VL) comprises a framework region (FR) and a complementarity determining region (CDR), wherein the complementarity determining region (CDR) comprises a CDR1, wherein a sequence thereof is QDISNY (SEQ ID NO:42), a CDR2, wherein a sequence thereof is YTS (SEQ ID NO:44), and a CDR3, wherein a sequence thereof is QQRLTPSPFT (SEQ ID NO:46); and the heavy-chain variable region (VH) comprises a framework region (FR) and a complementarity determining region (CDR), wherein the complementarity determining region (CDR) comprises a CDR1, wherein a sequence thereof is GGTFSNYW (SEQ ID NO:49), a CDR2, wherein a sequence thereof is TYRGHSDT (SEQ ID NO:51), and a CDR3, wherein a sequence thereof is ARNTALLDN (SEQ ID NO:53).

2. The single-chain antibody scFv as claimed in claim 1, wherein a sequence of the light-chain variable region (VL) is SEQ ID NO:4, and a sequence of the heavy-chain variable region (VH) is SEQ ID NO:3; or
a sequence of the light-chain variable region (VL) is SEQ ID NO:8, and a sequence of the heavy-chain variable region (VH) is SEQ ID NO:7; or
a sequence of the light-chain variable region (VL) is SEQ ID NO:12, and a sequence of the heavy-chain variable region (VH) is SEQ ID NO:11.

3. The single-chain antibody scFv as claimed in claim 1, wherein a sequence of the single-chain antibody scFv is SEQ ID NO:1; or SEQ ID NO:5; or SEQ ID NO:9.

4. A polynucleotide sequence for encoding the anti-BCMA single-chain antibody scFv as claimed in claim 1, wherein the polynucleotide sequence comprises a nucleotide sequence for encoding complementarity determining region sequences SEQ ID NO: 14, SEQ ID NO: 16 and SEQ ID NO: 18 of the light-chain variable region (VL); and a nucleotide sequence for encoding complementarity determining region sequences SEQ ID NO: 21, SEQ ID NO: 23, and SEQ ID NO: 25 of the heavy-chain variable region (VH); or
the polynucleotide sequence comprises a nucleotide sequence for encoding complementarity determining region sequences SEQ ID NO: 28, SEQ ID NO: 30 and SEQ ID NO: 32 of the light-chain variable region (VL); and a nucleotide sequence for encoding complementarity determining region sequences SEQ ID NO: 35, SEQ ID NO: 37, and SEQ ID NO: 39 of the heavy-chain variable region (VH); or
the polynucleotide sequence comprises a nucleotide sequence for encoding complementarity determining region sequences SEQ ID NO: 42, SEQ ID NO: 44 and SEQ ID NO: 46 of the light-chain variable region (VL); and a nucleotide sequence for encoding complementarity determining region sequences SEQ ID NO: 49, SEQ ID NO: 51, and SEQ ID NO: 53 of the heavy-chain variable region (VH).

5. The polynucleotide sequence as claimed in claim 4, wherein the polynucleotide sequence comprises a nucleotide sequence for encoding a sequence SEQ ID NO:4 of the light-chain variable region (VL); and a nucleotide sequence for encoding a sequence SEQ ID NO:3 of the heavy-chain variable region (VH); or
the polynucleotide sequence comprises a nucleotide sequence for encoding a sequence SEQ ID NO:8 of the light-chain variable region (VL); and a nucleotide sequence for encoding a sequence SEQ ID NO:7 of the heavy-chain variable region (VH); or
the polynucleotide sequence comprises a nucleotide sequence for encoding a sequence SEQ ID NO:12 of the light-chain variable region (VL); and a nucleotide sequence for encoding a sequence SEQ ID NO:11 of the heavy-chain variable region (VH).

6. The polynucleotide sequence as claimed in claim 4, wherein the polynucleotide sequence is SEQ ID NO: 2; or SEQ ID NO: 6; or SEQ ID NO: 10.

7. An expression vector, wherein the expression vector comprises the polynucleotide sequence as claimed in claim 4.

8. A host cell, wherein the host cell comprises the expression vector as claimed in claim 7 and can express an anti-BCMA single-chain antibody scFv.

9. A pharmaceutical composition, wherein the pharmaceutical composition comprises the anti-BCMA single-chain antibody scFv as claimed in claim 1, and a pharmaceutically acceptable carrier, a diluent or an excipient.

10. A method for preparing the anti-BCMA single-chain antibody scFv as claimed in claim 1, wherein the method comprises: transforming an expression vector containing a polynucleotide sequence encoding the single-chain antibody scFv as claimed in claim 1 to an expression host cell, culturing, and performing mass expression and purification of the anti-BCMA single-chain antibody scFv.

11. The method as claimed in claim 10, wherein the expression vector is a pComb3XSS vector, and the host cells is an Escherichia coli XL1-Blue strain or a TG1 strain.

12. A method for immunologically detecting BCMA, comprising:
under a condition sufficient for an antigen-antibody binding action to occur, contacting a sample with the anti-BCMA single-chain antibody scFv as claimed in claim 1 to form an immune complex; and
detecting the presence of the immune complex, the presence of the immune complex indicates the presence of BCMA.

13. The method as claimed in claim 12, wherein the sample is from a subject with Multiple Myeloma.

14. The method as claimed in claim 12, wherein in the single-chain antibody scFv, a sequence of the light-chain variable region (VL) is SEQ ID NO:4, and a sequence of the heavy-chain variable region (VH) is SEQ ID NO:3; or
a sequence of the light-chain variable region (VL) is SEQ ID NO:8, and a sequence of the heavy-chain variable region (VH) is SEQ ID NO:7; or
a sequence of the light-chain variable region (VL) is SEQ ID NO:12, and a sequence of the heavy-chain variable region (VH) is SEQ ID NO:11.

15. The method as claimed in claim 12, wherein in the single-chain antibody scFv, a sequence of the single-chain antibody scFv is SEQ ID NO:1; or SEQ ID NO:5; or SEQ ID NO:9.

16. An immunotherapy method for Multiple Myeloma, comprising
administrating a therapeutic effective amount of the anti-BCMA single-chain antibody scFv as claimed in claim 1 for a subject suffering from Multiple Myeloma.

17. The method as claimed in claim 16, wherein in the single-chain antibody scFv, a sequence of the light-chain variable region (VL) is SEQ ID NO:4, and a sequence of the heavy-chain variable region (VH) is SEQ ID NO:3; or
a sequence of the light-chain variable region (VL) is SEQ ID NO:8, and a sequence of the heavy-chain variable region (VH) is SEQ ID NO:7; or
a sequence of the light-chain variable region (VL) is SEQ ID NO:12, and a sequence of the heavy-chain variable region (VH) is SEQ ID NO:11.

18. The method as claimed in claim 16, wherein in the single-chain antibody scFv, a sequence of the single-chain antibody scFv is SEQ ID NO:1; or SEQ ID NO:5; or SEQ ID NO:9.

19. The pharmaceutical composition as claimed in claim 9, wherein in the single-chain antibody scFv, a sequence of the light-chain variable region (VL) is SEQ ID NO:4, and a sequence of the heavy-chain variable region (VH) is SEQ ID NO:3: or
a sequence of the light-chain variable region (VL) is SEQ ID NO:8, and a sequence of the heavy-chain variable region (VH) is SEQ ID NO:7; or
a sequence of the light-chain variable region (VL) is SEQ ID NO:12, and a sequence of the heavy-chain variable region (VH) is SEQ ID NO:11.

20. The pharmaceutical composition as claimed in claim 9, wherein in the single-chain antibody scFv, a sequence of the single-chain antibody scFv is SEQ ID NO:1; or SEQ ID NO:5; or SEQ ID NO:9.
